(19) United States
(12) Patent Application Publication
(10) Pub. No.: US 2022/0246231 A1
(43) Pub. Date: Aug. 4, 2022

(54) METHOD FOR CONSTRUCTING MODEL FOR PREDICTING DIFFERENTIATION EFFICIENCY OF IPS CELL AND METHOD FOR PREDICTING DIFFERENTIATION EFFICIENCY OF IPS CELL

(71) Applicants: SHIMADZU CORPORATION, Kyoto-shi, Kyoto (JP); KYOTO UNIVERSITY, Kyoto-shi, Kyoto (JP)

(72) Inventors: Makoto WATANABE, Kyoto-shi, Kyoto (JP); Taka-Aki SATO, Kyoto-shi, Kyoto (JP); Junya TOGUCHIDA, Kyoto-shi, Kyoto (JP)

(73) Assignees: SHIMADZU CORPORATION, Kyoto-shi, Kyoto (JP); KYOTO UNIVERSITY, Kyoto-shi, Kyoto (JP)

(21) Appl. No.: 17/623,777

(22) PCT Filed: Jul. 9, 2019
(86) PCT No.: PCT/JP2019/027225

§ 371 Date: Dec. 29, 2021

(51) Int. Cl. G16B 5/00; C12N 5/077; G16B 25/10; G16B 40/20

(52) U.S. Cl. CPC G16B 5/00; C12N 5/0655; C12N 2506/45; G16B 40/20; G16B 25/10

(57) ABSTRACT

A plurality of metabolites contained in culture supernatants of a plurality of iPS cell clones whose differentiation efficiency into chondrocytes or neural crest cells is known is quantified, and a relationship between the measured values of the plurality of metabolites obtained by the quantification and the differentiation efficiencies is subjected to a multivariate analysis, and a model for predicting a differentiation efficiency of iPS cells is constructed. Furthermore, the plurality of metabolites contained in a culture supernatant of a test cell group including a single type of iPS cell clones is quantified, and the values, obtained by the quantification, are applied to the model, thereby predicting a differentiation efficiency of the test cell group into chondrocytes or neural crest cells. This makes it possible to predict the differentiation efficiency of iPS cells into chondrocytes or neural crest cells in a short time.

### TECHNICAL FIELD

The present invention relates to a method for non-invasively predicting a differentiation efficiency of undifferentiated iPS cells (induced pluripotent stem cells), and particularly relates to a method for predicting a differentiation efficiency of iPS cells into chondrocytes and the like.

### BACKGROUND ART

As a differentiation pathway from iPS cells to chondrocytes, as shown in FIG. 20, a pathway in which iPS cells are differentiated into chondrocytes through neural crest cells (NC cells) has been known. As a method for finding iPS cell lines having a high differentiation efficiency into chondrocytes from among a plurality of iPS cell lines produced from human, there hitherto has been a method in which each iPS cell line is induced to differentiate into chondrocytes, and then expression levels of cartilage-related genes are measured in each cell (a shaded arrow in FIG. 20), or a method in which each cell is subjected to an immunohistochemical staining after a differentiation induction, whereby expression levels of cartilage-related proteins are measured.

According to such methods, however, it is necessary to differentiate the produced iPS cells into chondrocytes once, and thus they have a problem in which it takes a long period of time to measure the differentiation efficiency.

As a method in lieu of the methods described above, a method is known in which iPS cells are induced to differentiate into NC cells, and then an expression level of CD271, which is a cell surface marker protein of the NC cell, is measured in each cell (a white arrow in FIG. 20). When the cells after the differentiation induction into NC cells are stained with fluorescent antibodies and subjected to a quantitative analysis by a fluorescence flow cytometry, the cells after the differentiation induction are divided into a cell population having a low expression level of CD271 (CD271^{low+} NC cells) and a cell population having a high expression level (CD271^{high+} NC cells) (FIG. 21). When each of these cells is further induced to differentiate into chondrocytes, it has been found that cells that have been induced to differentiate from the CD271^{high+} NC cell population have a higher expression level of cartilage-related genes than that of cells that have been differentiated from the CD271^{low+} NC cells (Non Patent Literatures 1 and 2). It is possible, accordingly, to consequently select iPS cells having a high differentiation efficiency into cartilage by measuring an expression level of CD271 in each cell after performing the differentiation induction from the iPS cells into NC cells, and selecting iPS cell lines having a high percent of CD271^{high+} NC cells.

### CITATION LIST

### Non Patent Literature

Non Patent Literature 1: Umeda K. et al., Stem Cell Reports, 2015 Apr. 14; 4 (4), p. 712-26
Non Patent Literature 2: Fukuta M. et al., PLOS ONE, 2014 9 (12)

### SUMMARY OF INVENTION

### Technical Problem

According to the method for measuring the expression level of the marker protein CD271 after the differentiation induction into NC cells, as described above, the time necessary for measuring the differentiation efficiency can be remarkably shortened as compared with the method for measuring the expression level of the cartilage-related gene (or the cartilage-related protein) after the differentiation induction into the chondrocytes. Even in this case, however, at least about 8 days is necessary for differentiation induction into NC cells from iPS cells, and thus further shortening of the period has been required.

The present invention has been made in view of the points described above, and an object of the invention is to provide a method capable of predicting the differentiation efficiency of the iPS cells into the chondrocytes (or NC cells)(hereinafter, sometimes simply referred to as “differentiation efficiency”) in a short period of time.

### Solution to Problem

A method for constructing a model for predicting a differentiation efficiency of iPS cells according to the present invention, which has been made to solve the problems described above, includes:

collecting a culture supernatant from each of a plurality of iPS cell clones whose differentiation efficiency into chondrocytes or neural crest cells is known; quantifying a plurality of metabolites contained in each culture supernatant; subjecting the results obtained from the quantitation to a multivariate analysis to make a mathematical formula for predicting a differentiation efficiency of the iPS cells into the chondrocytes or neural crest cells from the quantitative values of the plurality of metabolites; and constructing a prediction model consisting of the mathematical formula.

Further, a method for predicting a differentiation efficiency of iPS cells according to the present invention, which has been made to solve the problems described above, includes: collecting a culture supernatant from a test cell group including a single type of iPS cell clones; quantifying a plurality of metabolites contained in the culture supernatant; and applying the resulting quantified values of the plurality of metabolites to the prediction model constructed as above, whereby a differentiation efficiency of the test cell group into chondrocytes or neural crest cells is predicted.

In the method for constructing a model for predicting a differentiation efficiency of iPS cells and the method for predicting a differentiation efficiency of iPS cells according to the present invention, it is desirable that the plurality of metabolites include 2-aminoethanol, 2-deoxyglucose, 2-hydroxyisocaproic acid, 2-hydroxyisovaleric acid, 2-methyl-3-hydroxybutyric acid, 4-aminobutyric acid, acetoacetic acid, cadaverine, dihydroxyacetone, fructose, galacturonic acid, gluconic acid, glutamic acid, glycine, isobutyrylglycine, lysine, lyxose, malic acid, mesaconic acid, methylsuccinic acid, mevalonic lactone, monostearyl, proline, psicose, succinic acid, tagatose, threitol, and threonine.

In the method for constructing a model for predicting a differentiation efficiency of iPS cells and the method for predicting a differentiation efficiency of iPS cells according to the present invention, the plurality of metabolites may further include at least one metabolite selected from 2′-deoxyuridine, 2-hydroxy-3-methyl valeric acid, 2-hydroxybutyric acid, 2-ketoadipic acid, 3-aminopropanoic acid, 3-hydroxydodecanedioic acid, allose, asparagine, citrulline, galactose, glucaric acid, glucosamine, glucose, maleic acid, mandelic acid, sorbitol, sorbose, sucrose, thymine, and xylitol.

The present invention further provides a method for acquiring neural crest cells having a high differentiation efficiency into cartilage.

That is, a method for acquiring neural crest cells having a high differentiation efficiency into cartilage according to the present invention includes: subjecting one or a plurality of test cell groups, each including a single type of iPS cell clones, to a prediction of a differentiation efficiency by the method of the present invention; subjecting one or a plurality of test cell groups that are predicted to have a high differentiation efficiency from the results of the prediction described above, to a differentiation induction into neural crest cells; and then subjecting the test cell groups that have been subjected to the differentiation induction, to a cell sorting using antibodies against CD271 protein, whereby cells having a higher expression level of the protein than a predetermined threshold level are sorted out.

### Advantageous Effects of Invention

According to the method for constructing a model for predicting a differentiation efficiency of iPS cells and the method for predicting differentiation efficiency of iPS cells according to the present invention, it is possible to predict a differentiation efficiency of the iPS cells into chondrocytes or NC cells based on metabolites contained in a culture supernatant of the undifferentiated iPS cells. It is not necessary, accordingly, that the iPS cells are induced to differentiate into the chondrocytes or NC cells as is done conventionally, and thus it is possible to reduce the time and effort required to find the differentiation efficiency.

### BRIEF DESCRIPTION OF DRAWINGS

FIG. 1 is a conceptual diagram illustrating a method for constructing a model for predicting a differentiation efficiency of iPS cells according to one embodiment of the present invention.

FIG. 2 is a flowchart for explaining a method for predicting a differentiation efficiency of iPS cells in the same embodiment.

FIG. 3 is a conceptual diagram showing procedures in a case in which cell groups having a high differentiation efficiency are specified from a plurality of iPS cell groups whose differentiation efficiencies are unknown.

FIG. 4 is a conceptual diagram illustrating a method for acquiring NC cells having a high differentiation efficiency into chondrocytes from iPS cell groups whose differentiation efficiencies are determined as high according to a prediction model.

FIG. 5 is a graph showing the percent of CD271^{high+} NC cells in a reference cell group after a differentiation induction into NC cells.

FIG. 6 is a graph showing the percent of CD271^{high+} NC cells in each of a cell group having a high differentiation efficiency (Good Clones) and a cell group having a low differentiation efficiency (Poor Clones).

FIG. 7 shows names of metabolites showing 1.5 folds or more of amount variation between cell groups having a high differentiation efficiency and cell groups having a low differentiation efficiency, and fold changes of the metabolites.

FIG. 8 shows names of metabolites showing a significant amount variation between cell groups having a high differentiation efficiency and cell groups having a low differentiation efficiency, and fold changes and p-values of the metabolites.

FIG. 9 shows score plots showing results of an OPLS analysis of a first metabolite profile.

FIG. 10 shows score plots showing results of an OPLS analysis of a second metabolite profile.

FIG. 11 is a table showing a coefficient given to a measured value of each metabolite in a first prediction model.

FIG. 12 is a table showing a coefficient given to a measured value of each metabolite in a second prediction model.

FIG. 13 is a graph showing R2 values and Q2 values in a cell group having a high differentiation efficiency (Good Clones) and a cell group having a low differentiation efficiency (Poor Clones) in the first prediction model.

FIG. 14 is a graph showing R2 values and Q2 values in a cell group having a high differentiation efficiency and a cell group having a low differentiation efficiency in the second prediction model.

FIG. 15 is a graph showing results of a permutation test for the first prediction model.

FIG. 16 is a graph showing results of a permutation test for the second prediction model.

FIG. 17 is a graph showing the percent of CD271^{high+} NC cells in a cell group for verification after a differentiation induction into NC cells.

FIG. 18 is a graph showing results obtained by applying a metabolite profile of a cell group for verification to the first prediction model.

FIG. 19 is a graph showing results obtained by applying a metabolite profile of a cell group for verification to the second prediction model.

FIG. 20 is a conceptual diagram for explaining a conventional method for measuring a differentiation efficiency into chondrocytes from iPS cells.

FIG. 21 is a graph showing expression levels of a marker protein CD271 in NC cells, measured by using a fluorescence flow cytometry.

### DESCRIPTION OF EMBODIMENTS

The method for constructing a model for predicting a differentiation efficiency of iPS cells according to the present invention includes:

collecting a culture supernatant from each of a plurality of iPS cell clones whose differentiation efficiency into chondrocytes or neural crest cells is known; quantifying a plurality of metabolites contained in each culture supernatant; subjecting the results obtained from the quantification to a multivariate analysis to make a mathematical formula for predicting a differentiation efficiency of iPS cells into chondrocytes or neural crest cells from the quantitative values of the plurality of metabolites; and constructing a prediction model consisting of the mathematical formula.

Further, the method for predicting a differentiation efficiency of iPS cells according to the present invention includes:

collecting a culture supernatant from a test cell group including a single type of iPS cell clones; quantifying a plurality of metabolites contained in the culture supernatant; and applying the resulting quantified values of the plurality of metabolites to the prediction model constructed as above, whereby a differentiation efficiency of the test cell group into chondrocytes or neural crest cells is predicted.

As a method for quantifying the plurality of metabolites contained in the culture supernatant, for example, quantitative analyses using a gas chromatograph mass spectrometer (GC-MS), a liquid chromatograph mass spectrometer (LC-MS), or a capillary electrophoresis mass spectrometer (CE-MS) can be preferably used. Other than the quantitative analyses using such a mass spectrometry, for example, a method may be used in which a sample obtained by subjecting a culture supernatant to a given pre-treatment is flown into a column of a liquid chromatography (LC) apparatus together with an eluent, components separated and eluted from the column are detected by using a detector such as an ultraviolet-visible spectrometer or an infrared spectrometer, and a plurality of metabolites contained in the sample are quantified from the results detected.

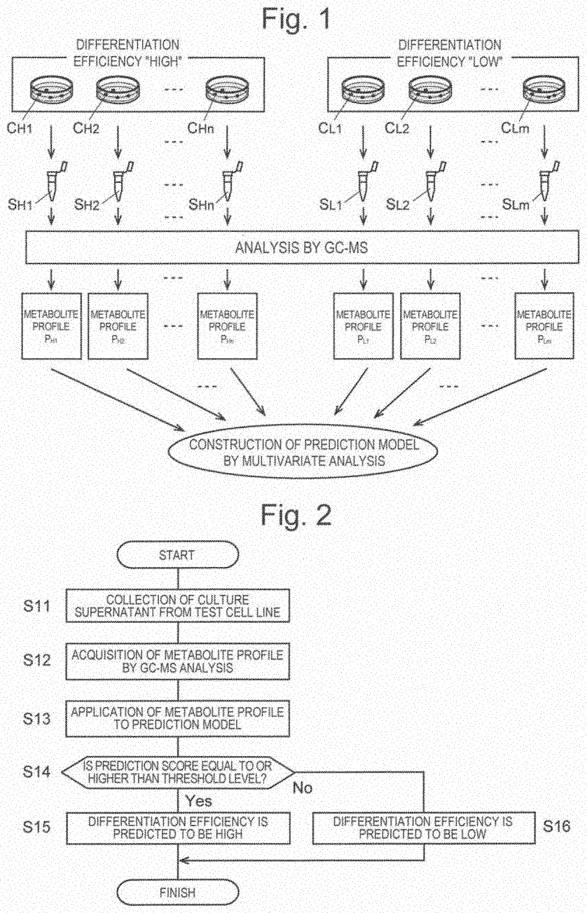

Procedures for creating a model for predicting a differentiation efficiency of iPS cells according to one embodiment of the present invention will be described with reference to FIG. 1. First, as reference cell groups, a plurality of iPS cell lines C_{H1}, C_{H2}, . . . C_{Hn} whose differentiation efficiency into chondrocytes or NC cells has been previously known to be high, and a plurality of iPS cell lines C_{L1}, C_{L2}, . . . C_{Lm} (n and m are each an integer of 2 or more, and hereinafter the same) whose differentiation efficiency has been known to be low, are prepared, and culture supernatants S_{H1}, S_{H2}, . . . S_{Hn} and culture supernatants S_{L1}, S_{L2}, . . . S_{Lm} are collected from each undifferentiated reference cell group. The culture supernatants S_{H1}, S_{H2}, . . . S_{Hn} and the culture supernatants S_{L1}, S_{L2}, . . . S_{Lm} are subjected to a quantitative analysis using GC-MS to acquire metabolite profiles P_{H1}, P_{H2}, . . . P_{Hn} and metabolite profiles P_{L1}, P_{L2}, . . . P_{Lm} for reference cell groups C_{H1}, C_{H2}, . . . C_{Hn} and reference cell groups C_{L1}, C_{L2}, . . . C_{Lm}, respectively. Here, the metabolite profile includes at least identifiers of the plurality of metabolites contained in the culture supernatant, and quantitative values of the metabolites. The “identifier” of a metabolite refers to a name, a number, or a symbol unique to each compound, and is typically a compound name, but may be, for example, m/z (mass-to-charge ratio) of a peak of the compound on a mass spectrum. The “quantitative value” refers to a value indicating the abundance of each metabolite in the culture supernatant, and can be, for example, an intensity of a detection signal of the compound, obtained by using a GC-MS detector, a concentration of the compound in the culture supernatant, calculated from the intensity, or the like. After that, the metabolite profiles P_{H1}, P_{H2}, . . . P_{Hn} acquired for the reference cell groups C_{H1}, C_{H2}, . . . C_{Hn} having a high differentiation efficiency and the metabolite profiles P_{L1}, P_{L2}, . . . P_{Lm} acquired for the reference cell groups having a low differentiation efficiency are subjected to a comparative analysis using a multivariate analysis to construct a prediction model.

For example, assuming that each metabolite profile includes data of metabolites A, B, C . . . , and that the measured values of the abundance of each metabolite are defined as [A], [B], [C] . . . , then a prediction model for distinguishing between iPS cells having a high differentiation efficiency and iPS cells having a low differentiation efficiency is as follows:

Prediction Score=i+a[A]+b[B]+c[C]

Here, for example, a model for predicting a differentiation efficiency of the present invention can be constructed by setting the constant term i and coefficients a, b, c . . . in the mathematical formula so that the differentiation efficiency is determined to be high when the prediction score described above is equal to or higher than a predetermined threshold T.

It is possible to preferably use orthogonal partial least square (OPLS) as a method of multivariate analysis used for constructing the prediction model described above, and it is also possible to use an analysis method such as a partial least squares regression (PLS) or a principal component analysis (PCA).

Next, working procedures of the method for predicting a differentiation efficiency of iPS cells according to one embodiment of the present invention will be described with reference to the flowchart of FIG. 2. First, culture supernatants are collected from iPS cell clones (a test cell group) whose differentiation efficiency is unknown, cultured in an undifferentiated state (Step S11), and the culture supernatants are subjected to a quantitative analysis using GC-MS to acquire metabolite profiles (Step S12). After that, the metabolite profiles are applied to a prediction model, which has been created in advance (Step S13), and whether or not the resulting prediction score is equal to or higher than a predetermined threshold value is determined (Step S14). When the prediction score is equal to or higher than the threshold level (Yes in Step S14), it is determined that the test cell group has a high differentiation efficiency (Step S15), and when the prediction score is lower than the threshold level (No in Step S14), it is determined that the test cell has a low differentiation efficiency (Step S16).

It is desirable that the plurality of metabolites, used for constructing the prediction model and predicting the differentiation efficiency of the test cells, include at least 2-aminoethanol, 2-deoxyglucose, 2-hydroxyisocaproic acid, 2-hydroxyisovaleric acid, 2-methyl-3-hydroxybutyric acid, 4-aminobutyric acid, acetoacetic acid, cadaverine, dihydroxyacetone, fructose, galacturonic acid, gluconic acid, glutamic acid, glycine, isobutyrylglycine, lysine, lyxose, malic acid, mesaconic acid, methylsuccinic acid, mevalonic lactone, monostearin, proline, psicose, succinic acid, tagatose, threitol, and threonine.

In addition, the plurality of metabolites, used for constructing the prediction model and predicting the differentiation efficiency of the test cell, may further include at least one metabolite selected from 2′-deoxyuridine, 2-hydroxy-3-methyl valeric acid, 2-hydroxybutyric acid, 2-ketoadipic acid, 3-aminopropanoic acid, 3-hydroxydodecanedioic acid, allose, asparagine, citrulline, galactose, glucaric acid, glucosamine, glucose, maleic acid, mandelic acid, sorbitol, sorbose, sucrose, thymine, and xylitol.

The method for predicting the differentiation efficiency of iPS cells according to the present invention can be preferably utilized, for example, in a case in which clones having a high differentiation efficiency into chondrocytes or NC cells are selected from among a large number of iPS cell clones. Even if iPS cell clones have a high differentiation efficiency, the differentiation efficiency may be decreased due to aging while the culturing is continued. When one iPS cell clone is used over a long period of time, the method for predicting the differentiation efficiency according to the present invention can also be used for a quality evaluation of the clone at each time point (confirmation of whether or not the differentiation efficiency is deteriorated).

In addition, as described in Non Patent Literatures 1 and 2, it has been found that chondrocytes differentiated from an NC cell population having a high expression level of the cell surface marker protein CD271 of the NC cells have a higher expression level of cartilage-related genes than that of those differentiated from an NC cell population having a low expression level of CD271. When the iPS cell clones, whose differentiation efficiency is predicted to be high by the method for predicting the differentiation efficiency described above, are induced to differentiate into NC cells, and cells having a high expression level of CD271 are sorted out from the differentiation-induced cells, then it is possible to acquire NC cells having a high differentiation efficiency into chondrocytes in a high efficiency.

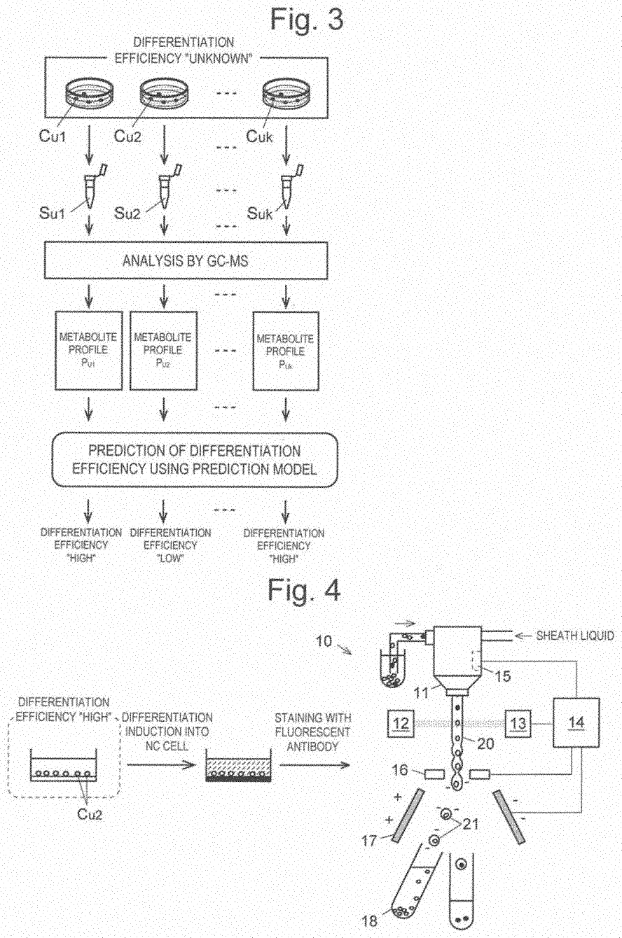

Specifically, first, as shown in FIG. 3, from a plurality of test cell groups C_{U1}, C_{U2}, . . . C_{Uk} (k is an integer of 2 or more, hereinafter the same), each including a single type of iPS cell clones, culture supernatants S_{U1}, S_{U2}, . . . S_{Uk} are collected respectively, they are subjected to a quantitative analysis by GC-MS to create metabolite profiles P_{U1}, P_{U2}, . . . P_{Uk}, and the metabolite profiles P_{U1}, P_{U2}, . . . P_{Uk} are applied to a prediction model previously created whereby the differentiation efficiency is predicted. One or a plurality of test cell groups (for example, C_{U2}), predicted to have a high differentiation efficiency resulting from above, are induced to differentiate into NC cells (FIG. 4), the test cell group C_{U2}, which has been subjected to the differentiation induction, is stained with fluorescent antibodies against the cell surface marker protein CD271, and then NC cells having a high expression level of CD271 (that is, the expression level of CD271 is equal to or higher than a predetermined threshold value) are sorted out by using a cell sorter 10 utilizing a fluorescence flow cytometry.

In the cell sorter 10, the test cells stained with the fluorescent antibodies are discharged from a nozzle 11 along the flow of the sheath liquid (sheath flow 20). At this time, the sheath flow 20 is irradiated with laser light emitted from a laser light source 12, and the fluorescence emitted from each cell by the irradiation of the laser light is detected by a detector 13. The detection signals from the detector 13 are sent to a control/data processing unit 14, and an amount of antigens present on the cell surface (that is, the expression level of CD271) is obtained based on the intensity of the fluorescence detected. A vibrator 15, provided in the nozzle 11, ultrasonically vibrates the nozzle 11, whereby the sheath flow 20 is changed into liquid droplets from the middle (below the irradiation position of the laser light). Furthermore, a charge-applying unit 16, provided below the nozzle 11, applies charges to the sheath liquid immediately before the sheath liquid containing the target cells (that is, the cells whose expression level of CD271 is higher than the predetermined threshold value) attempts to form the liquid droplets. As a result, charged liquid droplets 21 containing the target cells are generated, and the charged liquid droplets 21 are drawn to a deflection electrode plate 17 provided below the charge-applying unit 16, and collected in a recovery container 18. Here, the explanation is made citing a method using a cell sorter in which the target cells are sorted out by charging liquid droplets as an example, but a cell sorter having any method may be used.

### EXAMPLES

In Example, 14 types of human iPS cell lines, 201B2, 201B7, 414C2, 451F3, 409B2, TIG118-4f1, 604A1, 606A1, 610B1, 665A1, 703A1, 1503-4f1, TIG107-4f1, and TIG120-4f1 were used as reference cell groups for constructing a prediction model.

[Determination of Differentiation Efficiency by Known Method]

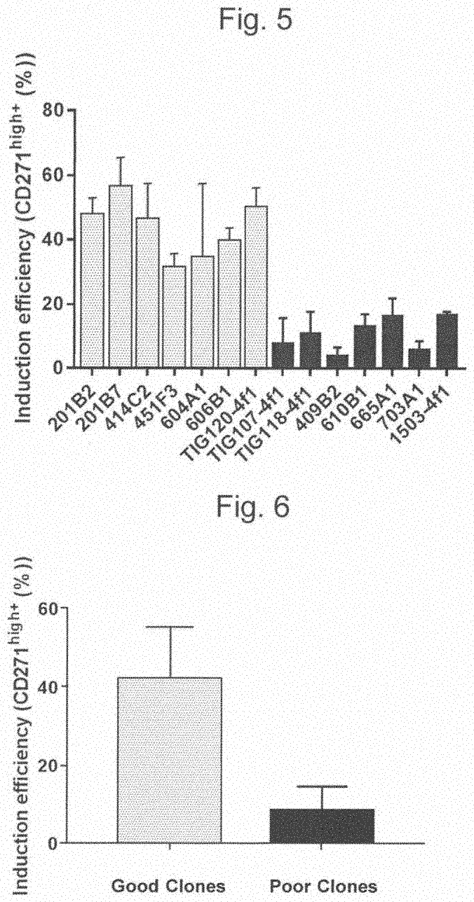

First, iPS cells of each reference cell group, cultured on feeder cells using a medium for iPS/ES cells (manufactured by Reprocel), were re-seeded on a culture dish coated with Matrigel (Matrigel Growth Factor Reduced, manufactured by Coming Incorporated) (passage ratio 1:5), and cultured in a feeder-free medium (mTeSR1, manufactured by Veritas Inc.) for 1 week. After that, the differentiation induction into neural crest cells was performed by culture in a NC differentiation medium for 6 days. The NC differentiation medium had a composition including 10 uM of SB431542, 450 uM of 1-thioglycerol, 170 uM of ascorbic acid-2 phosphate, 20 ug/mL of insulin, 100 ug/mL of human holo-transferrin, 2 mM of Glutamax-1, 37% of Iscove's Modified Dulbecco's Medium (IMDM) 2% CD lipid concentrate, and 9.4% of chemically defined medium (CDM) base (all final concentrations), and the CDM base was prepared by dissolving 5 g of bovine serum albumin in Ham's F12 Nutrient Mixture liquid and adding 127 mL of IMDM and 3 mL of a penicillin/streptomycin solution to the solution. After the differentiation induction, each reference cell group was stained with fluorescent antibodies against CD271, which were a cell surface marker protein of NC cells, and the percent of cells having a high expression level of CD271 (CD271^{high+} NC cells) was determined by using a fluorescent flow cytometry, and the resulting value was taken as a differentiation efficiency of each reference cell group. In the fluorescence flow cytometry, those whose intensity of fluorescence derived from the fluorescent antibody was equal to or higher than the predetermined threshold value were counted as the “CD271^{high+} NC cells” (such a threshold value may be decided by measuring a standard sample including only fluorescent antibodies, or may be decided based on a distribution when the fluorescence intensity in the target sample is formed into a histogram; in addition, the method for deciding a threshold value is not limited as long as it is set to such an extent that “CD271^{high+} NC cells” can be specified). As a result, as shown in FIG. 5, it was confirmed that the reference cell groups were divided into 7 cell line groups having a high differentiation efficiency (cell groups including a 20% or more of CD271^{high+} NC cells) and 7 cell line groups having a low differentiation efficiency (cell groups including less than 20% of CD271^{high+} NC cells). It was further confirmed that there was a statistically significant (p<0.001) difference in the differentiation efficiency (FIG. 6), when the differentiation efficiencies of the cell groups having a high differentiation efficiency and those of the cell groups having a low differentiation efficiency were subjected to a comparative analysis by a t-test.

[Acquisition of Metabolite Profile]

Next, a culture supernatant was recovered from a culture medium in which each reference cell group was cultured in an undifferentiated state, and each metabolite in the culture medium was quantified by an analysis using a gas chromatograph mass spectrometer (GC-MS) to acquire a metabolite profile. Specific procedures will be described below.

First, iPS cells, cultured on feeder cells, were re-seeded on a culture dish coated with Matrigel (Matrigel Growth Factor Reduced, manufactured by Coming Incorporated) (passage ratio 1:5), and cultured in a feeder-free medium (mTeSR1, manufactured by Veritas Inc.) for 1 week. After 1 week, the medium was recovered, then centrifuged at 3,000×g for 5 minutes, and then the supernatant was recovered as a medium metabolite sample.

In addition, the cells after removing the medium were washed with a PBS (phosphate buffered saline) solution, to which 1 mL of a papain solution was added, and then a cell suspension was recovered using a cell scraper. The cell suspension was allowed to stand at 60° C. overnight, then centrifuged at 15,000×g for 5 minutes, and a supernatant of the suspension was recovered as a DNA quantitative sample.

To 200 uL of the culture medium metabolite sample solution were added 800 uL of ice-cooled methanol and 1 uL of a ribitol solution (7.2 nmol/uL), and the mixture was allowed to stand at −20° C. for 30 minutes. After that, a supernatant was recovered by centrifugation at 10,000×g for 5 minutes, and then the sample solution was dried and solidified using a centrifugal concentrator. To the dried and solidified sample were added 80 uL of anhydrous pyridine and 40 uL of an MSTFA (N-methyl-N-TMS-trifluoroacetamide) solution, re-dissolution was performed, and then the solution was allowed to stand at 30° C. for 30 minutes. After the reaction, 1 uL of the sample solution was injected into GC-MS to acquire a profile of each metabolite.

Separately, DNA in the DNA quantitative sample was quantified using a Pico-Green reagent (manufactured by ThermoFisher Scientific), and the profile of each metabolite acquired by the analysis using GC-MS was divided by the internal standard MS peak intensity of the ribitol and the total amount of DNA, whereby the data normalization was performed.

[Selection of Candidate Metabolite to be Used for Model Construction]

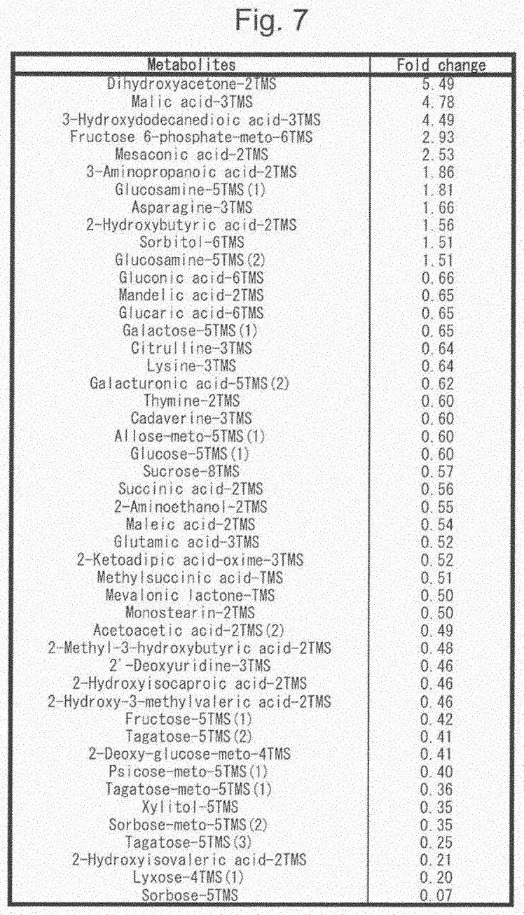

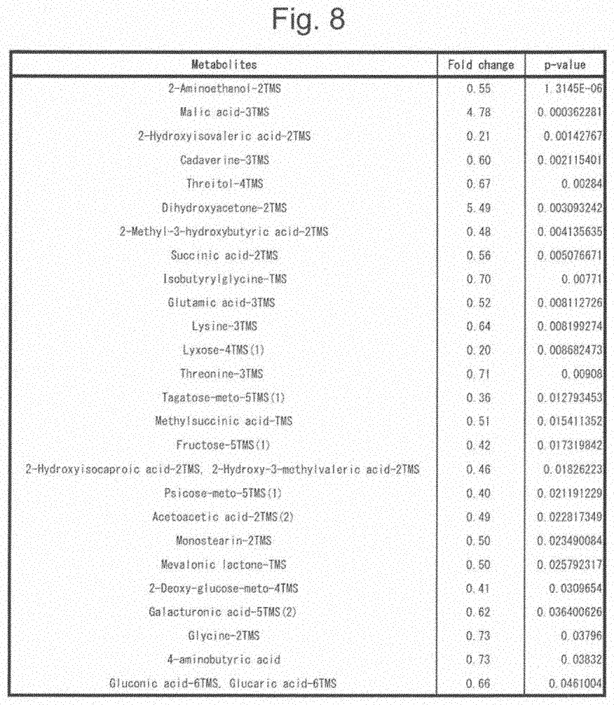

The acquired metabolite profiles were compared between the cell groups having a high differentiation efficiency and the cell groups having a low differentiation efficiency; as a result, it was confirmed that 47 types of metabolites had an amount variation of 1.5 times or more (FIG. 7). On the other hand, for 29 types of metabolites, the amount variation was found in statistical significance (p<0.05 by a significant test by a t-test) (FIG. 8). Note that “-nTMS”, “-nTMS(m)”, “-meto-nTMS”, “-meto-nTMS(m)”, or “-oxime-nTMS” (n and m are natural numbers) included in the metabolite names in FIG. 7 and FIG. 8 are derived from the reagent for GC added at the time of the analysis by GC-MS; for example, all of “Tagatose-meto-5TMS(1)”, “Tagatose-5TMS(2)”, and “Tagatose-5TMS(3)” in FIG. 8 are derived from the same metabolite (tagatose) in the medium.

[Study of Metabolite Profile]

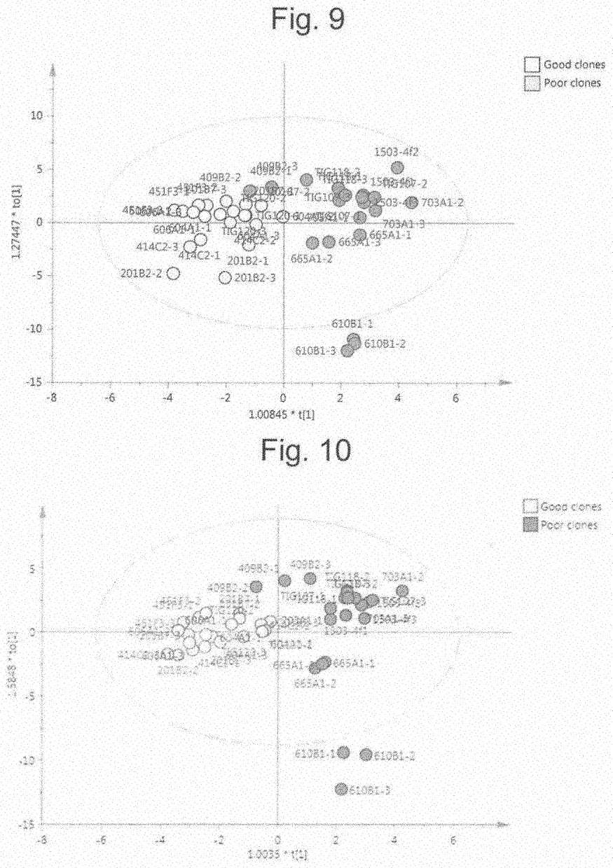

Using a multivariate analysis software SIMCA13, manufactured by Umetrics, multivariate analyses were performed on the metabolite profiles of the 47 types of metabolites (hereinafter referred to as “first metabolite profile”) and the metabolite profiles of the 29 types of metabolites (hereinafter referred to as “second metabolite profile”) using an OPLS method. As a result, score plots, as shown in FIG. 9 and FIG. 10, were obtained. As is clear from these figures, in any score plot, white circles indicating the cell groups having a high differentiation efficiency (Good Clones) and gray circles indicating the cell groups having a low differentiation efficiency (Poor Clones) showed different distributions, and it was suggested that a model (prediction model) for identifying between the cell groups having a high differentiation efficiency and the cell groups having a low differentiation efficiency can be constructed using either the first metabolite profiles or the second metabolite profiles.

[Construction of Prediction Model]

By instructing the construction of a prediction model using the first metabolite profiles on SIMCA13, a prediction model represented by the following formula (1) (hereinafter referred to as “first prediction model”) was obtained.

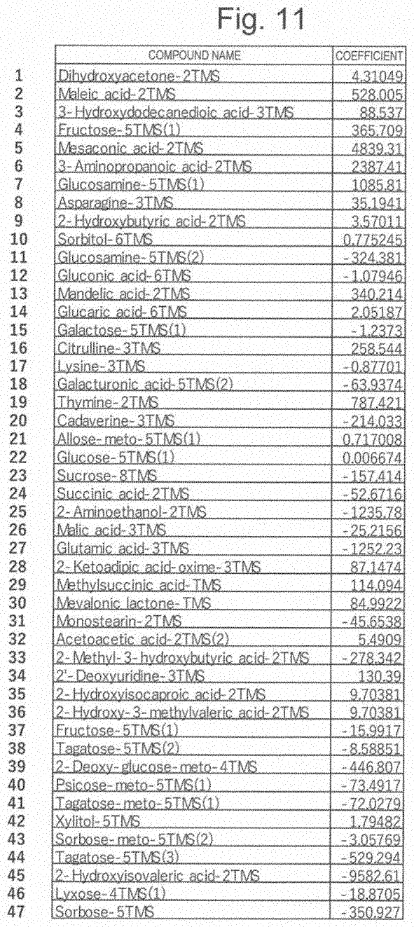

A constant (0.845284)+integral value of ((a coefficient for each metabolite (see FIG. 11))×(a measured value of each metabolite)) (1)

Similarly, by instructing construction of a prediction model using the second metabolite profiles on SIMCA13, a prediction model represented by the following formula (2) (hereinafter referred to as “second prediction model”) was obtained.

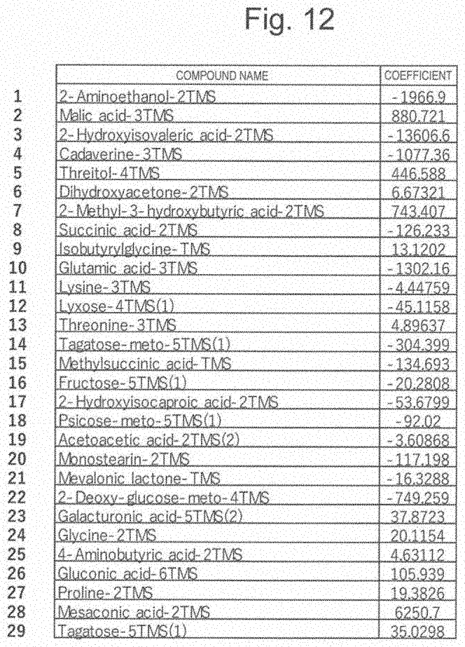

A constant (0.630201)+integral value of ((a coefficient for each metabolite (see FIG. 12))×(a measured value of each metabolite)) (2)

[Statistical Verification of Prediction Model]

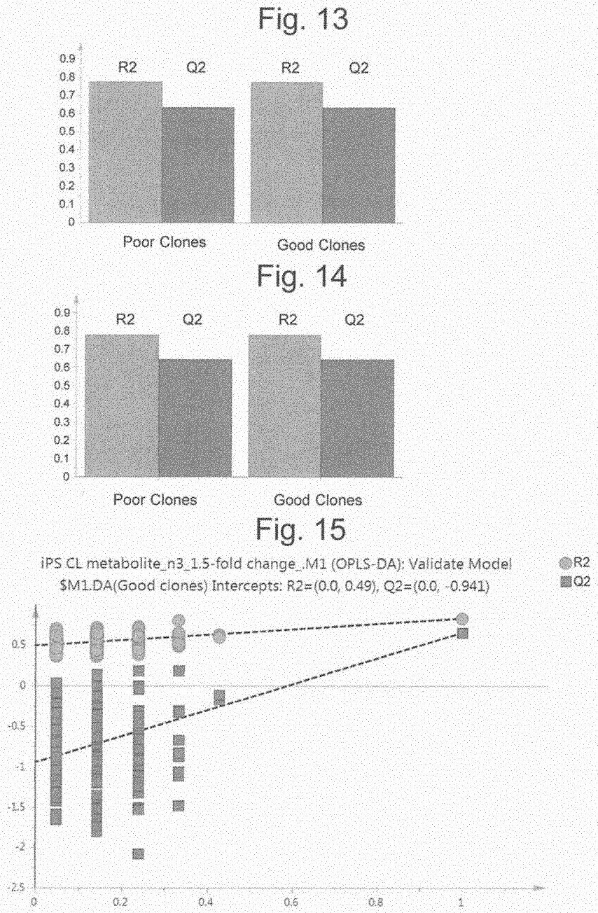

FIG. 13 shows the results of R2 values and Q2 values obtained for the first prediction model, and FIG. 14 shows the results of the R2 values and the Q2 values obtained for the second prediction model. The R2 value refers to an index indicating a degree of fitness of the model to the data used for the model construction, and it can be said that the degree of fitness of the model is higher as this value is closer to 1. The Q2 value refers to an index indicating fitness (predictability) of the model to unknown data, and when this value is 0.5 or more, it can be said that the predictability of the model is high. As illustrated in FIG. 13 and FIG. 14, it was confirmed that both the R2 values and the Q2 values of the first prediction model and the second prediction model were all high values.

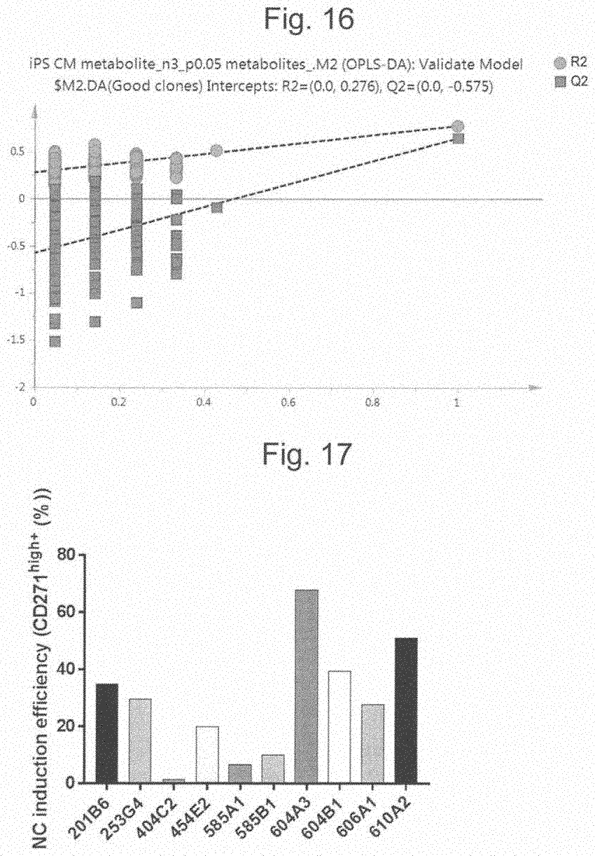

Furthermore, FIG. 15 and FIG. 16 show the results of testing the first prediction model and the second prediction model by a permutation test. In these figures, the vertical axis represents the R2 value or the Q2 value. The horizontal axis represents a frequency of data exchange, and the higher the data exchange frequency, the smaller the value of the horizontal axis. As is clear from these figures, it was confirmed that these prediction models did not overfit only the data used for model construction, because a y-intercept of the Q2 straight line was negative in both models.

[Prediction of Differentiation Efficiency Using Prediction Model]

Next, each prediction model was verified by applying the first prediction model and the second prediction model described above to other iPS cell lines.

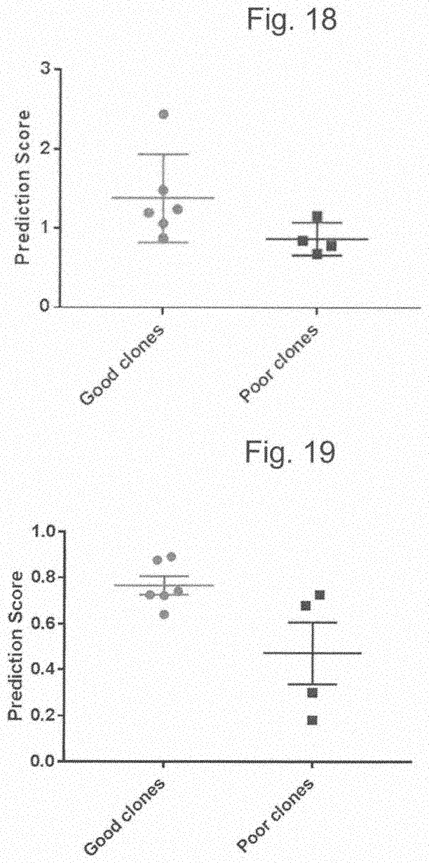

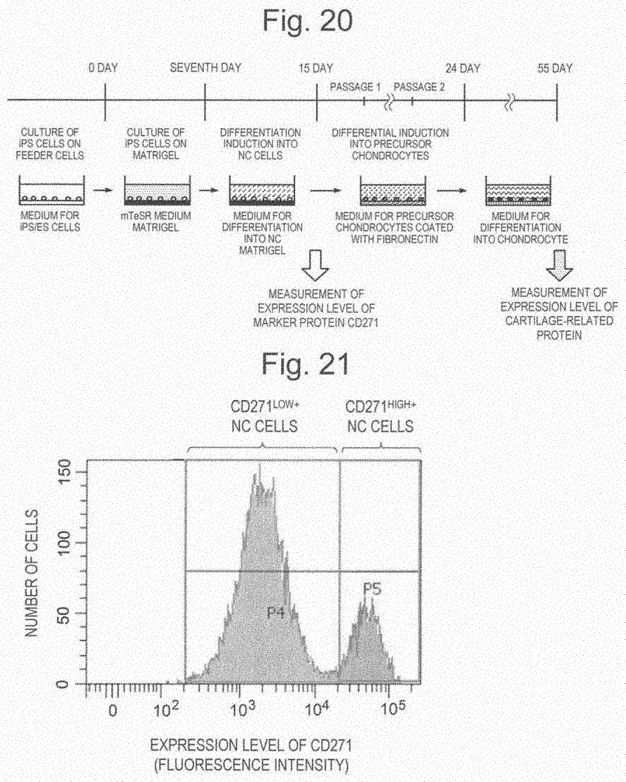

First, 10 types of human iPS cell lines, 201B6, 253G4, 404C2, 454E2, 585A1, 585B1, 604A3, 604B1, 606A1, and 610A2, different from those used for constructing the prediction model described above, were prepared as cell groups for verification, and analysis was performed for each cell group for verification by the fluorescence flow cytometry using antibodies against CD271 protein in the same manner as described above. As a result, it was confirmed that the cell groups for verification were divided into cell groups having a high differentiation efficiency (6 cell lines) and cell groups having a low differentiation efficiency (4 cell lines) (FIG. 17). Culture supernatants were recovered in the same manner as described above from culture media in which the cell groups for verification were cultured in the state wherein the iPS cells were undifferentiated, and a metabolite profile in each culture supernatant was acquired by using a GC-MS analysis. FIG. 18 shows a distribution of prediction scores obtained by applying measured values of the 47 types of metabolites shown in FIG. 7, among measured values of a plurality of metabolites included in each metabolite profile obtained as above, to the first prediction model (that is, substituting the measured values into (1) described above). FIG. 19 shows a distribution of prediction scores obtained by applying measured values of the 29 types of metabolites shown in FIG. 8 to the second prediction model (that is, substituting them into the formula (2) described above). As is clear from these figures, it was confirmed that the median of the prediction scores of the cell line having a high differentiation efficiency was higher than that of the prediction scores of the cell line having a low differentiation efficiency, regardless of which prediction model was used. From the above, it was confirmed that an actual differentiation efficiency of iPS cells could be predicted by these prediction models.

### REFERENCE SIGNS LIST

10 . . . Cell Sorter
11 . . . Nozzle
12 . . . Laser Light Source
13 . . . Detector
14 . . . Control/Data Processing Unit
15 . . . Vibrator
16 . . . Charge-Applying Unit
17 . . . Deflection Electrode Plate
18 . . . Recovery Container
20 . . . Sheath Flow
21 . . . Charged Liquid Droplet

1. A method for constructing a model for predicting a differentiation efficiency of iPS cells comprising: collecting a culture supernatant from each of a plurality of iPS cell clones whose differentiation efficiency into chondrocytes or neural crest cells is known; quantifying a plurality of metabolites contained in each culture supernatant; subjecting the results obtained from the quantification to a multivariate analysis to make a mathematical formula for predicting a differentiation efficiency of iPS cells into chondrocytes or neural crest cells from the quantitative values of the plurality of metabolites; and constructing a prediction model consisting of the mathematical formula.

2. A method for predicting a differentiation efficiency of iPS cells comprising: collecting a culture supernatant from a test cell group including a single type of iPS cell clones; quantifying a plurality of metabolites contained in the culture supernatant; and applying the resulting quantitative values of the plurality of metabolites to a prediction model constructed by a method according to claim 1, whereby a differentiation efficiency of the test cell group into chondrocytes or neural crest cells is predicted.

3. The method for predicting a differentiation efficiency of iPS cells according to claim 2, wherein the plurality of metabolites include 2-aminoethanol, 2-deoxyglucose, 2-hydroxyisocaproic acid, 2-hydroxyisovaleric acid, 2-methyl-3-hydroxybutyric acid, 4-aminobutyric acid, acetoacetic acid, cadaverine, dihydroxyacetone, fructose, galacturonic acid, gluconic acid, glutamic acid, glycine, isobutyrylglycine, lysine, lyxose, malic acid, mesaconic acid, methylsuccinic acid, mevaloic lactone, monostearin, proline, psicose, succinic acid, tagatose, threitol, and threonine.

4. The method for predicting a differentiation efficiency of iPS cells according to claim 3, wherein the plurality of metabolites further include at least one metabolite selected from 2′-deoxyuridine, 2-hydroxy-3-methyl valeric acid, 2-hydroxybutyric acid, 2-ketoadipic acid, 3-aminopropanoic acid, 3-hydroxydodecanedioic acid, allose, asparagine, citrulline, galactose, glucaric acid, glucosamine, glucose, maleic acid, mandelic acid, sorbitol, sorbose, sucrose, thymine, and xylitol.

5. A method for acquiring neural crest cells having a high differentiation efficiency into cartilage comprising: subjecting one or a plurality of test cell groups, each including a single type of iPS cell clones, to a prediction of a differentiation efficiency by a method according to claim 2; subjecting one or a plurality of test cell groups that are predicted to have a high differentiation efficiency from the results of the prediction above, to a differentiation induction into neural crest cells; and then subjecting the test cell groups that have been subjected to the differentiation induction, to a cell sorting using antibodies against CD271 protein, whereby cells having a higher expression level of the protein than a predetermined threshold level are sorted out.
